(19) United States
(12) Patent Application Publication
(10) Pub. No.: US 2018/0071050 A1
(43) Pub. Date: Mar. 15, 2018

(54) HOLDING ARM FOR MEDICAL PURPOSES HAVING A REMOVABLE OPERATING UNIT, AND AN OPERATING DEVICE THEREFOR

(71) Applicant: MEDINEERING GMBH, München (DE)

(72) Inventors: Stephan Nowatschin, München (DE); Maximilian Krinninger, Weßling-Oberpfaffenhofen (DE); Dominikus Gierlach, München (DE)

(21) Appl. No.: 15/561,048

(22) PCT Filed: Mar. 24, 2016
(86) PCT No.: PCT/EP2016/056570

§ 371 Date: Sep. 23, 2017

(30) Foreign Application Priority Data
Mar. 27, 2015 (DE) 10 2015 104 819.1

(51) Int. Cl. A61B 90/50; A61B 90/98; A61B 34/00

(52) U.S. Cl. CPC A61B 90/50; A61B 2090/0813; A61B 34/74; A61B 90/98

(57) ABSTRACT

The invention relates to a holding arm for medical purposes, the holding arm including at least a first and a second arm segment, where the first arm segment is connected to a first joint and the second arm segment is connected to a second joint, and where each joint can be released and locked by means of an releasing device. The holding arm also includes an operating unit for bringing the holding arm into a desired pose and a control unit which is coupled to the operating unit and the releasing device to transmit signals from the operating unit to the releasing device. According to the invention, an interface for receiving a key and a detecting means for detecting an identity of the key are provided, where the control unit is designed to control the releasing device according to the identity of the received key.

The invention relates to a holding arm for medical purposes, in particular for holding a surgical mechatronic assistance system and/or a surgical instrument, comprising a proximal end for attaching the holding arm to a base and a distal end for receiving a surgical mechatronic assistance system and/or a surgical instrument; at least one first and one second arm segment, wherein the first arm segment is connected to a first joint and the second arm segment is connected to a second joint, wherein each joint can be released and locked by means of a releasing device, an operating unit for bringing the holding arm into the desired pose, and a control unit which is coupled to the operating unit and the releasing device in order to transmit signals from the operating unit to the releasing device. The invention also relates to an operating unit for using with the holding arm.

Holding arms of the kind initially specified have long been known from the prior art and are specifically used in surgery to relieve an operator of static holding work. Such a holding arm is used to hold a mechatronic assistance system and/or a surgical instrument, for example a manipulator, an endoscope, a surgical clamp or the like. The holding arms initially specified have proved their usefulness for holding endoscopes, in particular. In endoscopic surgery, an operator generally operates an instrument with both hands, while an assistant holds the endoscope in order to make the operating area visible on a screen. Holding the endoscope over a protracted period is very tiring. Holding arms are increasingly used for that reason.

Such a holding arm is known from DE 195 26 915 B4, for example. The holding device for medical purposes disclosed therein has a connection member and a holder for surgical tools, as well as an arm arranged between the holder and the connection member. The arm is connected to the holder and to the connection member, or to an adjacent arm via a joint, and can be coupled to a pneumatically operable device for selectively locking and releasing the joints, wherein the device locks the joints by the action of a mechanical spring which exerts a braking force on the joint, and wherein the device can be pneumatically switched to a joint-releasing mode against the force of that spring. An actuator by means of which a valve can be opened is disposed on the holder at the proximal end of the arm, so that the separate joints of the arm can be adjusted. When the actuator is released, the valve is closed again, thus locking the joints.

A similar holding arm is disclosed in EP 1 958 587 B1. The holding arm disclosed therein likewise has a plurality of joints, and a touch-sensitive sensor for actuating the joints is provided. The sensor is disposed on the holding arm adjacent to the medical instrument, so that the operator comes into contact with the touch-sensitive sensor on gripping the medical instrument, as a result of which the joints of the holding arm are released.

The holding arm disclosed in DE 195 26 915 B4 and also the one disclosed in EP 1 958 587 B1 are used primarily as a kind of exoskeleton for the operator, so that the operator can rest on the holding arm during the operation and can release all the joints on gripping the medical instrument or when operating the actuator, so that the pose of the holding arm can be altered.

Another holding arm, adapted to hold an endoscope, is known from DE 10 2004 050 714 A1. The arm has a plurality of joints which can be closed pneumatically. The holding arm is connected to a foot-switch valve. When the foot-switch valve is operated, compressed air enters all the joints, thus releasing them.

One disadvantage, however, is that precise positioning of the mechatronic assistance system and/or surgical instrument disposed on the holding arm is difficult to achieve with the holding arm known from the prior art, and is strongly dependent on the skill of the operator. The precision of positioning is confined solely to the skills of the operator who spatially positions the distal end of the arm.

Another problem is that the aforementioned holding arms are either universal in design, or designed for a specific application. There are holding arms, for example, that can be used in any operation, in general, whatever the specific application, and there are holding arms that are specifically designed for a particular field of application, such as head surgery. In the former case, the problem is that these sometimes special functions for specialized applications are not provided, whereas the latter are of little or no use for other kinds of operation.

The object of the present invention is to provide a holding arm and an operating unit of the kind initially specified, which can be adapted in a simple and reliable manner to a respective operation being performed.

This object is achieved, in a holding arm of the kind initially specified, with the features of claim 1, in particular, that is, by an interface for receiving a key, and a detecting means for detecting the identity of the key, the control unit being designed to control the releasing device according to the identity of the received key.

Due to the control unit being designed to control the releasing device according to the identity of the received key, and preferably does control the releasing device according to the identity of the received key, the holding arm as a whole can be adapted to the special conditions of an operation in a particular field by selecting an appropriate key with a respective identity. The releasing device preferably has an active brake in each joint. Alternatively or additionally, the releasing device has a drive unit in each joint of the holding arm. The control unit is preferably coupled to the detecting means to receive the detected identity. This can be accomplished using a bus system inside the holding arm.

The holding arm has an operating unit which is designed to bring the holding arm into a desired pose and also being designed to release the associated joint upon contact between an operator and one of the first and second arm segments, in particular an operating unit arranged thereon. It is therefore preferred that the operating unit is adapted to release the first joint when contact occurs between an operator and the first arm segment and to release the second joint when contact occurs between an operator and the second arm segment. When an operator comes into contact with a respective arm segment, only the associated joint is preferably released. This makes it possible to move individual joints intuitively and thus to adjust the holding arm segment by segment and to bring it into a desired pose. By this means, positioning can be carried out with greater precision, because each segment can be separately adjusted incrementally. It is likewise possible to contact a plurality of segments at once, with the result that a plurality of joints can be released and thus adjusted simultaneously. This allows the holding arm to be brought into a desired pose in a simple manner, and in particular intuitively.

In addition to the first and second arm segments, further arm segments which are each associated in like manner with a respective joint are preferably provided. The arm segments themselves are substantially rigid and preferably rod-shaped. The expression “rod-shaped” here includes not only substantially straight arm segments, but also slightly or strongly curved arm segments. In such a holding arm, arm segment and joints always alternate, and the holding arm at the distal and at the proximal end can end with a joint or with a segment or with a connection member. The holding arm can be attached to a base with its proximal end. The base may alternatively be coupled securely to the arm, or the arm can be removed from the base. In one embodiment, the base is in the form of an operating table, and the holding arm can be coupled to an operating table. The holding arm can preferably be coupled to a standard rail provided on the operating table. Such standard rails are generally provided on operating tables, so a standard interface can be provided on the holding arm to couple it to the standard rail of an operating table. Normal operating tables are also assembled from separate segments. For coupling purposes, the segments have matching, generally manufacturer-specific coupling points on their front sides. The holding arm can preferably to attached to the operating table via such a coupling point. A manufacturer-specific adapter may be provided for that purpose at the proximal end. Alternatively, the base is provided as a separate apparatus, for example a stand which can be set up on the floor of an operating theatre. In another alternative, the base is configured as a holder which can be attached to a wall or ceiling of an operating theatre, for example.

The holding arm is preferably configured as a passive holding arm, so called, and for that reason has joints which are actively braked exclusively, but not driven joints as is often the case with robotic holding arms. Each joint is therefore releasable and lockable only, but cannot be driven. As a result, the holding arm is simple in design and does not need a complex controller in order to operate it.

The holding arm preferably has a first interface at its proximal end for connecting the holding arm to an energy source and to an external control unit for transmitting signals to and from the holding arm; a second interface at its distal end for coupling the holding arm to the assistance system in order to control the assistance system; and a transmission means which is arranged inside the holding arm and which connects the first interface to the second interface for transmitting energy and signals between the interfaces. The interfaces and also an internal supply system and/or data bus system are preferably provided in the form described in DE 10 2014 016 823, which is held by the present applicant and the entire disclosure of which is incorporated herein by reference.

Assistance systems within the meaning of the invention are understood to be any kind of mechatronic manipulators which are used in surgery, such as endoscopes, exoscopes, laparoscopes, trocars and the like. The second interface at the distal end of the holding arm is designed to couple mechanically with the assistance system in order to hold the latter in a defined position relative to the holding arm, and also to provide the other connections that are necessary, such as a connection for electrical energy and a connection for transferring signals, in particular control signals. A transmission means, which preferably has a bus system, is provided inside the holding arm. The transmission means also has means for transmitting electrical energy. Any cables that are required in order to transmit electrical energy and/or data from the first interface to the second interface are thus arranged inside the holding arm arranged and are thus protected during operation of the holding arm. At the first interface, means are also provided for coupling the holding arm to an energy source and to an external control unit, such as a computer and/or an OP system. This provides the holding arm with a greater range of possible applications, and allows it to be used in a versatile manner for different assistance systems. Safety is improved at the same time, because it is not necessary to attach additional cables or the like. Instead, the assistance system need only be connected to the second interface at the distal end, and the holding arm itself only has to be couplable via the first interface at the proximal end to an energy source and an external control unit.

In a first preferred embodiment of the invention, the operating unit has the interface for receiving a key, wherein the key is arranged on an operating unit and the interface is designed for receiving the operating unit in a reversibly releasable manner and for receiving control signals from the operating unit. The key is thus coupled to the operating unit and represents the identity of the operating unit. According to this embodiment, the control unit is preferably designed to control the releasing device according to the identity of the received operating unit.

In one advantageous variant, the key is provided in the form of a data key or as a software key. A data key includes coding, for example, and takes the form of a 64-bit key, for example. It can also have some other appropriate item of data, such as a password, an operator identifier, an identifier for an operation to be performed, a patient identifier, or the like. In this case, the interface is designed for wired or wireless reception of the data key and the detecting means has software means for decrypting the key. A software key preferably includes a software module containing program code which is received or embedded in a program by the control unit, or processed in a program by the control unit. Such a software module may contain program instructions for controlling the holding arm, the assistance system and/or equipment which is attached to the assistance system.

The operating unit of the holding arm preferably has a plurality of such interfaces, preferably at least one and preferably two interfaces on each arm segment. It is preferable that the holding arm is basically designed as disclosed in DE 10 2014 016 824, held by the present applicant, the operating unit according to the present invention having the contacting means of the holding arm according to DE 10 2014 016 824. In this way, the holding arm according to the present invention is designed to release the respective associated joint upon contact between an operator and one of the first and second arm segments, in particular an operating unit arranged thereon. With regard to the other characteristics of releasing individual joints by contacting the associated arm segment, reference is made to the disclosure in DE 10 2014 016 824, the entire contents of which are incorporated here by reference.

Two interfaces are preferably formed on each arm segment substantially opposite one other in relation to a longitudinal axis of the arm segment, so that two operating unit per arm segment can be arranged substantially opposite one another. This has the advantage that the operator does not have to contact the arm segment quite as exactly, but that it is sufficient if the operator's grip is substantially around the arm segment and thus comes into contact with the operating means.

According to another preferred embodiment, the control unit is designed to control the releasing device in such a way, depending on the identity of the received key, that when a first identity of a first key is detected, a first plurality of restrictions for the holding arm is provided, and that when a second identity of a second key is detected, a second plurality of restrictions for the holding arm is provided. Such a plurality of restrictions may consist, for example, in a restriction on the poses adopted by the holding arm, in the angular speed of individual or of all joints, in angular accelerations of individual or of all joints, or in a restriction of the jolt.

For example, the first identity of the first key received may specify that it is a key for abdominal surgery, so this key is selected by an operator who is handling an abdominal surgery case. A restriction on angular speeds of all the joints may be provided here, for example. However, if the identity of the key is one for head surgery, the angular speeds may be restricted even further to a very low value, and one or more poses may also be restricted. For example, it may be specified, based on detection of the identity, that a movement of the holding arm is possible in one plane only, and that all the poses that are outside that plane are not allowed and that the control unit controls the releasing device accordingly. That means that if a operator tries in such a case to move the holding arm beyond the permitted plane, the control unit will control the releasing device in such a way that the joints of the holding arm are not released and that such movement is prevented. As a result, the safety of the holding arm is significantly increased and the holding arm is flexibly adapted to the situation in particular fields of operation by selecting the appropriate key. Prior to the operation, the operator selects a key or a set of keys which are then arranged, before the operation begins, in the respective receptacle or the respective receptacles of the holding arm. The detecting means of the respective interface detects the identity of each key received at this interface and communicates the identity to the control unit. The control unit processes that information accordingly and receives or calls a respective program, in particular a software program, which includes the restrictions and/or capabilities of the holding arm that are associated with the respective identities. Such a program can be pre-stored and can include pre-stored restrictions for a plurality of identities. Prior to an operation, an operator can store a particular identity with a particular set of restrictions in order to prepare the holding arm for the planned operation. Alternatively or additionally, the detecting means may likewise detect such restrictions when it detects the identity of the key, meaning that the restrictions are stored in the key and that this information is transferred to the control unit. During the operation, the operator no longer needs to take care that the holding arm is not mistakenly moved beyond the desired restrictions, since this is blocked by means of the control unit by the arm itself, due to the detected identity. A wrong movement, resulting in injury to the patient, is thus prevented.

In another preferred embodiment, the detecting means has an optical detecting means for optically detecting the identity of the key. Such an optical detecting means is preferably provided in the form of a camera. A camera which can detect every key is preferably provided for the entire holding arm. Alternatively and preferably, a camera which can detect the identity of the respective key received at the interface is provided adjacent to a respective interface on each arm segment. Such a camera is preferably coupled to an analyzer unit which detects the identity of the received key by means of image processing.

The analyzer unit may be arranged in the holding arm or outside the holding arm. The analyzer unit is preferably connected to the camera or cameras via a bus system in the holding arm.

In one preferred development of the invention, the optical detecting means is designed to detect colour coding and/or structural coding of the identity of the key. It is preferred, for example, that the key is colour coded. A particular colour may thus be specified for each surgical application, for example. Keys for head surgery are red, for example, whereas keys for abdominal surgery are green. This has the simultaneous advantage that the coding is also clearly recognizable and understandable for the operator, so he can easily check by himself whether the correct key was selected. Structural coding is provided, alternatively or additionally. Such structural coding can have an external shape, for example, such as oval or rectangular, or can have a surface structure or specially formed protrusions such as a protrusion with two, three or four prongs, for example. Another conceivable kind of coding is multicolour coding, such as a barcode consisting of black and white stripes. Such a barcode may also be provided in colour, for example red and white stripes, green and white stripes, and the like. A QR code designed in such a way in preferred. The advantageous here is that the operator can easily classify the code on the basis of the colour of the key, in addition to which the barcode or QR code can transfer extended information, such as a first set of restrictions, to the detecting means.

In another preferred embodiment, the detecting means has a receiver for receiving electromagnetic waves, wherein the electromagnetic waves represent the identity of the key. In this embodiment, an RFID transponder, for example, is provided on the key or as the key and a matching RFID reader is provided in the region of the interface on the respective arm segment, so that when the key is received, the RFID transponder is within the field of the reader and is read by means of the reader. The RFID reader, for its part, is preferably coupled to the control unit, for example via a bus system arranged in the holding arm. The advantage here as well is that information, such as restrictions or the like, can be sent in addition to just the identity of the key by means of the RFID transponder. Alternatively or additionally thereto, the key has an NFC chip via which the key and a matching reader arranged in the holding arm can communicate with each other. If the key also has its own microcontroller with the respective energy supply and storage means, or is connected to same, then it is also possible for information and data to be sent via an NFC chip from the holding arm to the key, and to devices connected to it, such as the operating unit.

In another preferred embodiment, the detecting means has an electronic interface for receiving the identification signal of the key. Such an electronic interface may take the form, for example of a USB interface, a cinch interface or the like. According to such an embodiment, the key preferably has a matching plug which can be inserted into such an interface. The identity of the inserted key can be transmitted in a simple manner to the holding arm via the latter also. At the same time, such an interface also provides a kind of mechanical coupling, and when the key is inserted into the interface it also provides mechanical feedback via the connection.

According to another preferred embodiment, the detecting means has an electromechanical lock for receiving a plurality of keys, the control unit being designed to control the releasing device according to the keys that are received. A stepped recess in the interface on the holding arm is preferably provided, for example, into which a matching projection on the key can be inserted. A plurality of electrical switches are arranged in the stepped recess, which close an electrical contact depending on the shape of the projection on the key. The identity of the key can be recognized from the combination of electrical contacts that are closed. This is a particularly simple way of encoding and detecting the identity. Such an electromechanical lock may be designed as a pin lock, the individual pins of which are moved according to the shape of the key and this movement being detected by the detecting means, with the result that the identity of the key is detected. This is another option for simple mechanical coding, which also allows many variants, so that many keys can be encoded for many different operations and operational conditions.

According to another preferred embodiment, the holding arm has a sterile bag surrounding the holding arm, wherein the bag has a recess and/or can be penetrated and/or can be permeated by electromagnetic waves in the region of the interface in order to receive an operating unit in a reversibly releasable manner and/or to receive a key. It is possible in any of these ways to clad the holding arm with a sterile bag to begin with and then subsequently to arrange a respective key and/or a respective operating unit on the holding arm. This simplifies handling to a significant degree. The operating unit may be outside the bag yet still interact with the holding arm, so that the holding arm can be operated by the operating unit. The safety of the holding arm is improved as a result.

It is also preferred that the operating unit and/or the interface is arranged on the assistance system. Such an assistance system may be provided in the form of a manipulator which is designed to receive a kinematic module. A kinematic module can hold a medical instrument, for example, such as an endoscope or a biopsy needle, or the like. In this embodiment, the kinematic module preferably has the key, and the latter is received in the interface formed on the manipulator by arranging, and in particular by inserting the kinematic module on the manipulator. By detecting the identity of the key, it is then possible to detect, for example, whether the kinematic module is new or used, how many operating hours it has been in sue, or whether it has been sufficiently sterilized. Safety is significantly improved as a result.

According to a second aspect of the invention, the object referred to at the outset is achieved by an operating unit for a holding arm according to one of the preferred embodiments of a holding arm as described in the foregoing, wherein the operating unit has a key for the interface for receiving a key. The operating unit has the key and is preferably designed as a key. In addition to the key, the operating unit may have further elements and devices for operating the holding arm. Provided it is arranged at the interface for receiving the key, the key is detected by the detecting means, so the identity of the key is detected and the control unit controls the releasing device according to that identity.

It should be understood that the holding arm according to the first aspect of the invention and the operating unit according to the second aspect of the invention have common preferred embodiments and variants as specified in the dependent claims, in particular. The advantages of the invention are embodied, in particular, by a combination of the holding arm according to the first aspect of the invention and the operating unit according to the second aspect of the invention, when they interact with each other. Reference is made in that regard to the entire description above and to the advantages described therein, and both element, the holding arm and the operating unit, are claimed both independently of each other and also jointly as a system.

According to a first preferred embodiment of the operating unit, the latter has a base member, an input device with which the operator can enter a control signal, and an interface for transmitting the control signal to the holding arm, wherein the key specifies the identity of the operating unit and can be detected by means of the detecting means of the holding arm. According to this embodiment, the key encodes the identity of the operating unit. The operating unit preferably has a contact surface as described above and as disclosed in DE 10 2014 016 824. The operating unit, including the contact surfaces thereon, can thus be arranged on the holding arm, and in particular can be inserted in the latter, in order to allow the holding arm to be operated. Due to the operating unit also having, in addition to the key, the input device which is preferably designed as a touch-sensitive contact surface and with which the operator can enter a control signal, the input device along with the operating unit can be reversibly removed from the holding arm, so that the holding arm cannot be operated without the operating unit or operating units arranged thereon. This enhances safety, in turn. Furthermore, it is possible in this way to match the input device to a special field of surgery. For example, it may be necessary to provide a different kind of input device for the field of head surgery than for the field of abdominal surgery. The operating unit, including the input device, can also be sterilized separately, which is likewise conducive to safety.

In one preferred development of the invention, the key contains visual coding. According to this embodiment, the key may take the form of a coloured area provided on a top side of the operating unit. The key may also be provided in the form of a barcode or a QR code. A combination of a colour-coded area and a barcode or QR code is preferred. It is further preferred that the key has a visually detectable structural coding, such as a surface structure, a shape, a contour or a protrusion having a specific shape.

According to another preferred embodiment, the key has a transmitter for transmitting electromagnetic waves. For example, the key has an RFID transponder which transmits electromagnetic waves representing the identity of the operating unit whenever it is within the electromagnetic field of an RFID reader. If the operating unit is arranged at the respective interface on the holding arm, the RFID transponder can be read by the reader and the identity of the operating unit can be established.

It is also preferred that the key has an electronic interface for transmitting an identification signal. According to such an embodiment, the key may be designed as a chip which is equipped with a USB connection, a cinch connection or the like. Other interfaces are also conceivable here.

According to another preferred embodiment, the key is provided in the form of a mechanically insertable key. The key preferably has a structured surface by means of which the identity of the operating unit is encoded. The key preferably has bitting which can be sensed by means of a pin lock on the holding arm and converted into an electronic signal.

In another preferred variant, the operating unit has an interface for an optical reflector for surgical navigation. Such reflectors are used by conventional OP navigation systems to identify individual instruments, equipment or specific points, and to integrate them into navigation. An operating unit having an interface for such a reflector is preferably arranged on each arm segment. In this way, each arm segment of the holding arm, on which an operating unit according to the second aspect of the invention is arranged, can be integrated into the navigation system. This is particularly preferred, because the operating unit is arranged outside a sterile bag that surrounds the holding arm. The interface for the reflector is also outside the bag, therefore, and the reflector may be arranged on the holding arms without further ado and without intruding on the bag.

It is further preferred that the operating unit comprises display devices, preferably illumination devices, for displaying a status of the operating unit and/or of the holding arm and/or of a mechatronic assistance system and/or of a surgical instrument attached to the holding arm. It is firstly preferred that the display devices, which are preferably provided in the form of illumination devices, display a status of the operating unit. Such a status may be that the operating unit is correctly connected to the interface of the holding arm. Another status may be that the operating unit has been correctly identified by the holding arm or by the detecting means of the holding arm and has been assigned a specific, previously known plurality of restrictions. The illumination devices are preferably in the form of coloured LEDs which indicate the status by lighting up and flashing. Such display devices are also preferably used to display a status of the holding arm. For example, the display devices indicate whether the holding arm is working correctly, or whether an error has been detected. They can also indicate any overloading of the holding arm, or a collision with a predetermined restriction of the operating area. Furthermore, the display devices preferably display the status of a mechatronic assistance system and/or surgical instrument connected to the holding arm. In this case also, the display devices can indicate whether the assistance system has been correctly coupled to the respective interface at the distal end of the holding arm.

For that purpose, the holding arm preferably has a bus system to which the assistance system is coupled and via which data are exchanged. Both the control unit and the detecting means, as well as the interface for the operating unit, are connected to this bus system. If the operating unit has respective communication means, such as an NFC chip, data and information can be exchanged between the operating unit and the holding arm, so that a status of the assistance system can be transmitted via the bus system and the NFC connection to the operating unit, which then displays the status of the assistance system by means of the display devices.

In another preferred embodiment, the operating unit has a screen for displaying data and/or a model of the patient. Such data may include, for example, the insertion depth of an endoscope, the suction pressure of a suction device, the weight forces acting on the distal end of the holding arm, or the like. All the data required to perform the operation can be displayed on the screen. It is also possible to display a model of the patient on the screen, showing in particular the position of the holding arm relative to the patient, or the position, relative to the patient, of an assistance system or instrument arranged at the distal end. The screen is close to the operating area as a result, so the surgeon can look in the direction of the operation area and see not only the operation area itself, but also the screen displaying the most important data. It is possible in this way to avoid the surgeon having to turn his head frequently in order to watch a screen located some distance away, which always involves the eyes having to adjust their depth of focus, which can quickly cause them to tire.

According to another preferred embodiment, the operating unit has a vibration module with which the operating unit can be made to vibrate. The operating unit is thus able to provide the person operating it with tactile feedback. The vibration module can be designed in such a way, for example, that it vibrates for a short moment, for example for half a second or a quarter of a second, if the operator touches a contact surface of an operating unit in such a way that a signal to release a joint is transmitted from the operating unit to the control unit of the holding arm. Alternatively, the operating unit can be made to vibrate whenever the operator tries to carry out an action with the holding arm that is not permitted, for example when he tries to bring the holding arm into a pose which is not allowed due to the detected identity of the operating unit. This increases the level of safety and in addition provides the surgeon with feedback about the actions he performs.

In one particularly preferred embodiment, the input device of the operating unit has a touch-sensitive surface area for controlling the holding arm. The touch-sensitive surface is preferably designed in the same way as the contact surface according to DE 10 2014 016 824 and as additionally described below. According to this embodiment, it is preferable that the operating unit also has sensors which detect touch. The touch-sensitive surface is designed as a touchpad or touch display, for example.

Alternatively, the touch-sensitive surface is provided with a fluid body. The latter has a cavity, for example, which is sealed with an elastic layer on one outwardly facing side.

There is a gel or some other fluid in the cavity, as well as one or more pressure sensors. If a surgeon now touches the elastic surface, the pressure inside the cavity increases, which is then detected by the one or more pressure sensors. The resultant signal is used as a control signal which is passed on to the control unit in the holding arm by means of the bus system in the holding arm. A joint is then released or locked on the basis of this control signal. Such a system with a fluid chamber has the advantage that the individual components are inexpensive and that the operating unit designed in this way can be easily sterilized. Furthermore, such a system is less prone to error and is easily scalable. Regardless of which side of the elastic layer the surgeon touches, a corresponding signal is generated by the one or more pressure sensors.

According to another preferred embodiment, the operating unit has an electronic coil for surgical navigation. The coil is preferably connected to the arm via an interface, and the interface of the holding arm is preferably connected at the proximal end to an OP system and via the latter to the other OP equipment in the operating theatre, such as, more specifically, an electromagnetic navigation system for the operating theatre. This allows single segments of the holding arm, or the holding arm in its entirety, to be navigated in an electromagnetic navigation environment.

According to another preferred embodiment, the input device has a capacitive sensor which is configured to detect a change in the distance of the operating unit from the holding arm and to convert said change into an electrical signal. It is not necessary in this regard that the entire operating unit change its distance from the operating unit. It is also sufficient if this applies to just a part of it, for example a surface. The input device has a surface, for example, which is elastic and which interacts with the capacitive sensor. When pressure is applied to this elastic surface, it is slightly deformed in the direction of the holding arm, with the result that the capacitive sensor generates a signal which is passed via the in and the internal bus system of the holding arm to the control unit, which releases or locks a joint on the basis of that signal. Such a variant has the advantage that the input device has a sealed surface, which makes it easier to sterilize.

Preferably, or alternatively, the operating unit has one or more additional sensors on its underside facing the holding arm, or on a mechanical pin with which the operating unit is connected to the holding arm. A matching electronic module is provided in the holding arm. In combination with the sensor and the outer casing of the holding arm, the operating unit forms a means of capacitive distance measurement. Such a variant allows the slightest movement of the operating unit on top of the holding arm to be detected and converted into a control signal.

If an operating unit having the key is arranged at a respective interface of the holding arm, or if a plurality of operating units is arranged, in particular two per arm segment, the result is a system which combines the advantages described in the foregoing and the effects described in DE 10 2014 016 824. In particular, the operating unit may be designed to release the associated joint according to the intensity of contact. What is meant by intensity here is a pressure and/or force which is applied by the operator. It is possible in this way for the operator to control a degree of freedom with the force that he applies when gripping. It is thus conceivable and preferred that the associated joint is only partially released when the intensity of contact is low, so that the arm segment can be moved only slowly and against a resistance. Whenever the intensity is high and thus when the grip is strong, the joint is opened completely, so the arm segment can be moved with substantially no resistance. The joint can also be partially released by releasing it intermittently in different frequencies. It is preferable that the first joint be disposed at a proximal end of the first arm segment and that the second joint be disposed at a proximal end of the second arm segment. Each segment has a proximal and a distal end, the proximal end of the arm segment being the end which, in the direction of the arm, is proximal to the proximal end of the holding arm, and the distal end of the arm segment is the end that is oriented to the distal end along the holding arm.

The joints of the holding arm preferably have brakes by means of which the joints can be released and locked. The holding arm is preferably in the form of a passive holding arm. The purpose of the brakes is to brake or prevent movement of the arm segments relative to each other, i.e. to brake or prevent any movement of the joints. If the brakes are released, the joints are released. In an idle state, the brakes are preferably biased in such a way that the joints are locked. It is particularly preferred that the brakes are designed as electromagnetic brakes and that they each comprise a permanent magnet which biases the brake into the locked state when no current is being supplied.

According to another preferred embodiment, the holding arm has six degrees of freedom. It is particularly preferred that the holding arm has seven degrees of freedom. Whereas six degrees of freedom are sufficient to reach any point in space, it is possible with seven degrees of freedom to reach any point with different poses, so the holding arm can always be oriented in such a way that the operating area is easily accessible, for example. For that reason, it is particularly preferred that the holding arm has seven degrees of freedom. According to one preferred embodiment, the holding arm has seven arm segments and seven joints, with each arm segment being assigned one joint. According to this embodiment, each joint preferably has one degree of freedom, so the holding arm has a total of seven degrees of freedom. It is also possible that each joint has two or more degrees of freedom, with joints having one degree of freedom being preferable on account of their stability. All the joints are preferably designed as rotary joints. It is preferable that some of the joints are designed as rotary joints and some as translational joints. When the joints are all designed as rotary joints, they are preferably disposed in the holding arm in such a way that axes of successive joints along the holding arm, from the proximal to the distal end of the holding arm, are perpendicular to each other. The holding arm preferably has a weight compensation means for at least partially supporting the weight of one or more arm segments of the holding arm when one or more joints are released.

Orientation indicators showing a basic pose of the holding arm may also be arranged on the arm segments.

It is also preferred that at least one cable duct is provided inside the arm segments to guide cables from the proximal to the distal end of the holding arm. In another preferred embodiment, the first arm segment, relative to the proximal end of the holding arm, has first mechanical coupling means for releasably coupling the holding arm to a second corresponding coupling means of an operating table. It is also preferred that the last arm segment at the distal end of the holding arm has a mechatronic interface for coupling the surgical mechatronic assistance system and/or the surgical Instrument to the holding arm. Such a mechatronic interface preferably has mechanical coupling means for holding the assistance system and/or the surgical instrument mechanically, and electronic interfaces for transmitting electrical energy and/or data or signals to the mechatronic assistance system.

The invention shall now be described in more detail with reference to four embodiments and with reference to the attached Figures, in which:

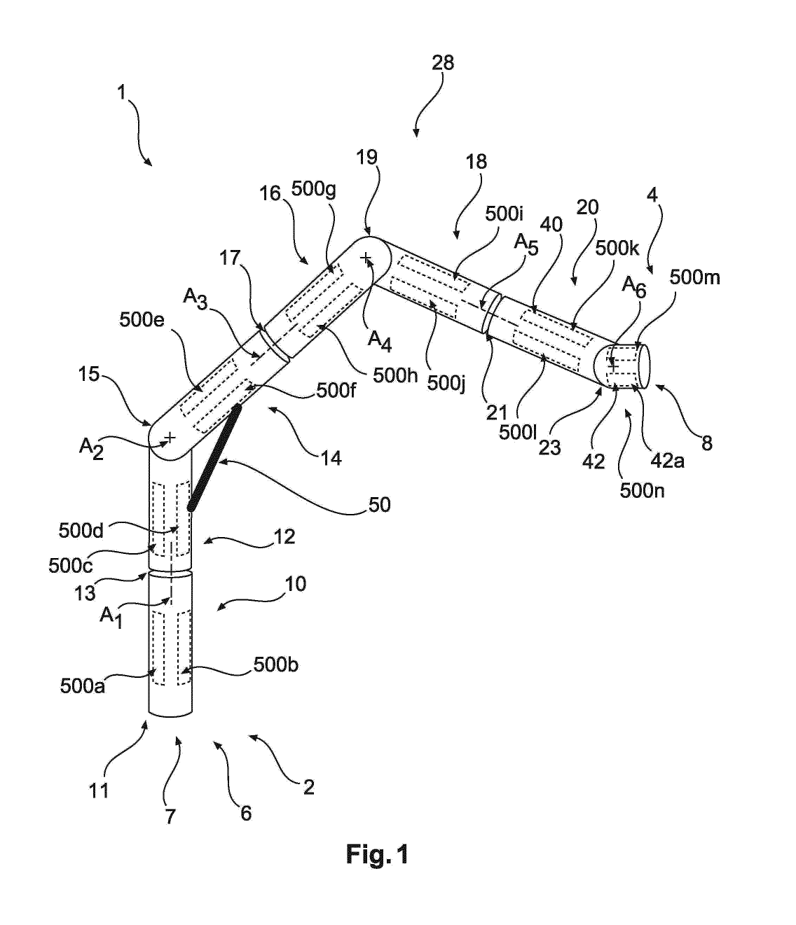

FIG. 1 shows a side view of a holding arm, in which the contacting means can be seen;

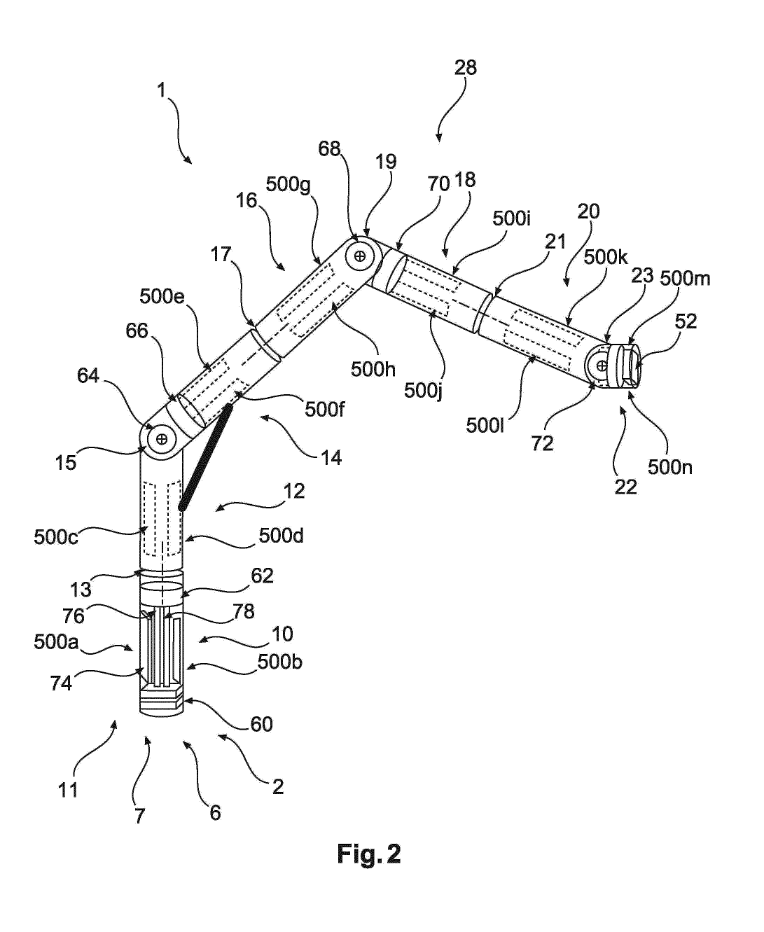

FIG. 2 shows a partly cutaway view of the holding arm shown in FIG. 1;

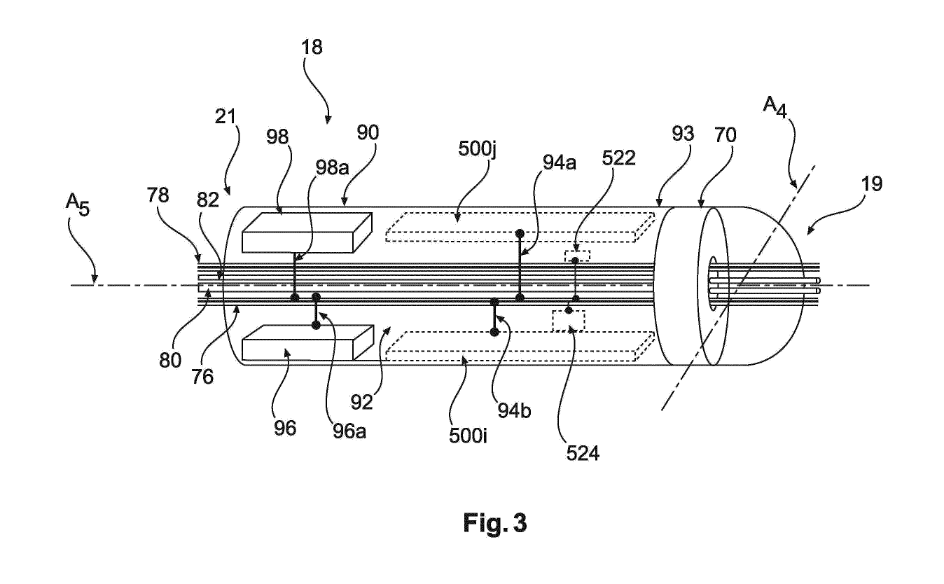

FIG. 3 shows a schematic view of the fourth arm segment;

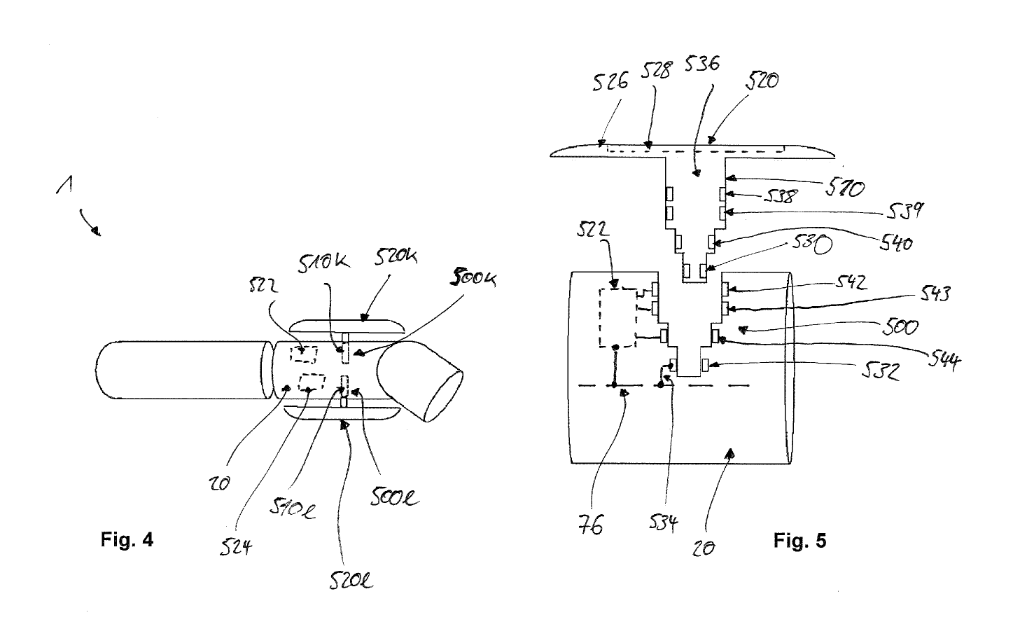

FIG. 4 shows three holding arm segments with an interface, a key and an operating unit;

FIG. 5 shows a holding arm segment with an interface and a key according to a first embodiment;

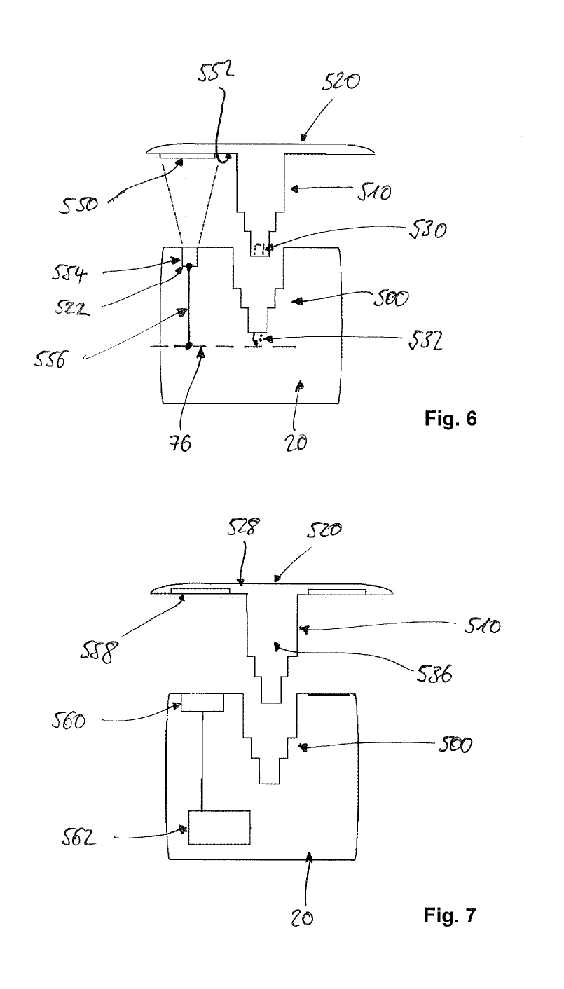

FIG. 6 shows a holding arm segment with an interface and a key according to a second embodiment;

FIG. 7 shows a holding arm segment with an interface and a key according to a third embodiment;

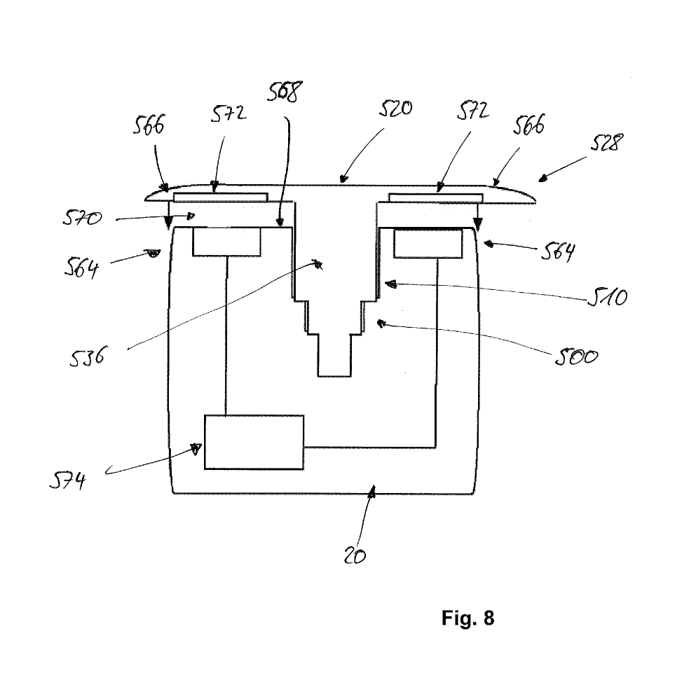

FIG. 8 shows a holding arm segment with an interface and a key, in which capacitive distance measurement is provided; and

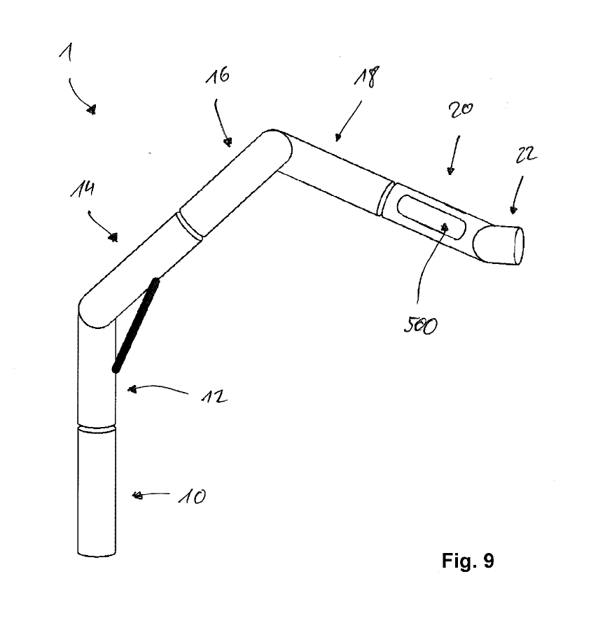

FIG. 9 shows a holding arm according to a second embodiment.

FIG. 1 shows a holding arm 1 for medical purposes, in particular for holding a surgical mechatronic assistance system and/or a surgical instrument. Holding arm 1 has a proximal end 2 and a distal end 4. At the proximal end 2, a first interface 6 and a mechanical interface 7 are formed. Interface 7 is used to attach holding arm 1 to a base, such as an operating table. Interface 6 is used to transfer energy and to couple holding arm 1 to an external control unit. At the distal end 4, a second interface 8 is provided via which it is possible to couple a mechatronic assistance system and/or a surgical instrument, such as a manipulator, to holding arm 1. A manipulator for holding and manipulating an endoscope is preferably disposed here.

The holding arm 1 according to FIG. 1 has seven arm segments 10, 12, 14, 16, 18, 20, 22, each of which is substantially rod-shaped and all of which, except for the last arm segment 22, are of substantially the same length. The seven arm segments 10, 12, 14, 16, 18, 20, 22 are each coupled to one another by means of joints 11, 13, 15, 17, 19, 21, 23, the zero-th joint 11 coupling holding arm 1 to the base (not shown in FIG. 1). In this embodiment, joints 13, 15, 17, 19, 21, 23 are all in the form of rotary joints each having one degree of freedom. According to this embodiment, the zero-th joint 11 is associated with the zero-th segment 10, the first joint 13 with the first arm segment 12, the second joint 15 with the second arm segment 14, the third joint 17 with the third arm segment 16, the fourth joint 19 with the fourth arm segment 18, the fifth joint 21 is associated with the fifth arm segment 20, and the sixth joint 23 is associated with the sixth arm segment 22. Joint 11 is designed as a translational joint, so that arm segment 10 can be extended telescopically in order to adjust the height of holding arm 1. Joints 13, 15, 17, 19, 21, 23 have respective pivot axes A₁, A₂, A₃, A₄, A₅, A₆, with respectively adjacent joints having pivot axes that are perpendicular to each other. This allows simple positioning of distal end 4 in space.

Holding arm 1 according to FIG. 1 also includes an operating means 28. By means of operating unit 28, holding arm 1 can be brought into a desired pose, operating unit 28 being adapted to release the associated joint upon contact between an operator and one of the seven arm segments. For that purpose, according to this embodiment, operating unit 28 has seven pairs of interfaces 500a-500n (collectively marked with reference sign 500) for receiving a key (cf. FIG. 5). One key can be received at each of these interfaces 500, and the interfaces can serve simultaneously as interfaces for fourteen operating units (cf. FIGS. 5-7).

In another embodiment, holding arm 1 has only one interface (500) for just one key 510 (cf. FIG. 9).

It can also be seen in FIG. 1 that holding arm 1 has a weight compensation means 50. In this embodiment, weight compensation means 50 has a gas spring element which is coupled to arm segment 14 and arm segment 12. Alternatively, the weight compensation means may also have a cable pull and/or a equilibrated counterweight. In the case of holding arm 1 as shown in FIG. 1, the strongest torque is exerted on joint 15 about its rotational axis A2. It is therefore preferred that precisely that joint 15 be supported by means of weight compensation means 50. Thus, when joint 15 is released by contacting arm segment 14, a weight acting upon arm segment 14 due to the other arm segments 16, 18, 20, 22 and a manipulator disposed at interface 8, is supported by weight compensation means 50 so that the distal end 4 does not “sag” immediately when segment 14 is gripped.

In addition to the elements of holding arm 1 already shown in FIG. 1, FIG. 2 shows brakes 60, 62, 64, 66, 68, 70, 72, by means of which joints 11, 13, 15, 17, 19, 21, 23 can be released and locked. Identical and similar elements are marked with the same reference signs as in FIG. 1, and reference is made in that respect to the entire description above.

A brake 60, 62, 64, 66, 68, 70, 72 is associated with each joint 11, 13, 15, 17, 19, 21, 23. Brake 60 is associated with joint 11, brake 62 with joint 13, brake 64 with joint 15, brake 66 with joint 17, brake 68 with joint 19, brake 70 with joint 21 and brake 72 with joint 23. All the brakes 60-72 are provided in the form of electromagnetic brakes and each comprise a permanent magnet which biases the brake into the locked state when no current is being supplied. The permanent magnet is designed in such a way that it can brake the respective joint on its own and so that the pose of holding arm 1 is held. In the zero-th arm segment 10, an electronic control unit 74 is provided. The latter is coupled via a bus system 76 (only shown in arm segment 10 in FIG. 2; cf. FIGS. 3 and 4) to all the interfaces 500a-500n of operating unit 28 and to all the brakes 60-72. In order to supply energy to brakes 60-72 and to interfaces 500a-500n, an energy supply line 78 is also provided, which can be coupled to an energy source via interface 6 at the proximal end 2 of holding arm 1.

FIG. 3 shows by way of example the fourth arm segment 18, in an enlarged, partly cutaway view. It should be realized that the other arm segments 10, 12, 14, 16, 20, 22 may be configured the same way.

Arm segment 18 has an arm segment body 90 (not shown in FIGS. 1 and 2; it should be understood that each arm segment 10-22 has such an arm segment body), which according to FIG. 3 is substantially rod-shaped and substantially cylindrical. Arm segment body 90 has a hollow space 92 inside, in which various elements such as brake 70 are arranged. Joints 19, 21 and the two pivot axes Aa, As of joints 19, 21, which interact with holding arm segment 18, are shown schematically in FIG. 3. Joint 19 is associated with holding arm segment 18 (cf. the description above referring to FIGS. 1 and 2). Arm segment body 90 has an outer surface 93 which is substantially cylindrical. Arm segment body 90 is made, for example, of a metal such as aluminum or titanium, an aluminum- or titanium-based alloy, or a composite fibre material such as GRP or CFRP, and is preferably of lightweight construction.

According to FIG. 3, arm segment 18 has interfaces 500i, 500j, which are part of operating unit 28 (cf. FIGS. 1 and 2). The two interfaces 500i, 500j are arranged substantially opposite one another in relation to axis A₅, so an operator who grips arm segment 18 comes into contact with both the operating units if the respective operating units, including the keys, are arranged at interfaces 500i, 500j.

The two interfaces 500i, 500j are coupled by means of lines 94a, 94b to bus system 76. Interfaces 500i, 500j are coupled via bus system 76 to the electronic control unit 74 (cf. FIG. 2) and via the latter to brake 70, so that brake 70 is released by operating unit 28 when an operator comes into contact with operating units that are accommodated in interfaces 500i, 500j.

In addition to bus system 76, an energy transmission system 78 and a cable channel 80 and a working channel 82 are arranged inside arm segment body 90. By means of energy transmission system 78, interfaces 500i, 500j and brake 70 are connected to an energy supply.

Alternatively or additionally, an electronics module 96 which is coupled to bus system 76 via a line 96a is disposed inside each arm segment. In such a case, interfaces 500i, 500j, which are connected via line 94a, 94b to data bus 76, interact only with electronics module 96, which converts the signals received by the operating units in interfaces 500i, 500j into a control signal for brake 70 and sends said control signal via bus system 76 to brake 70 in order to release joint 19. If such an electronics module 96 is disposed inside each arm segment, holding arm 1 has a substantially modular structure, and the individual arm segments 10-22 are independent of the electronic control unit 74 which is disposed in proximal arm segment 10.

Cable channel 80 is used to guide cables running from the proximal end 2 to the distal end 4 to supply interface 8, in particular. Working channel 82 is used to receives tubes or waveguides and the like as may be required by that particular kind of manipulator disposed at interface 8. If, for example, an endoscope is disposed at interface 8, a waveguide which can transmit an image recorded by an endoscopic camera is preferably guided through working channel 82. Working channel 82 is thus used to receive transmission means appropriate to the particular field of application.

There is also a sensor 98 disposed inside arm segment 18. A sensor is preferably disposed in each arm segment 10-22, and it should be understood that the sensors in arm segments 10, 12, 14, 16, 20 and 22 may be configured in the same way as sensor 98 in arm segment 18. Sensor 98 is preferably provided in the form of an acceleration sensor. By providing such an acceleration sensor in each arm segment, it is possible to determine the pose of holding arm 1 at any time. For that purpose, sensor 98 is coupled via line 98a to data bus 76, so that the data captured by sensor 98 are transmitted to the electronic control unit 74, which then determines the pose of holding arm 1 from all the sensor data from all the arm segments. By providing such a sensor 98, it is also possible to determine the absolute and relative position of an end effector or manipulator disposed at interface 8. If holding arm 1 is attached to an operating table, it is also possible to detect any movement of the operating table. If all the sensors in all the arm segments detect a movement in the same direction, this is an indication that the entire holding arm 1 has been moved while keeping its pose, for example by the operating table or a plate of the operating table having been rotated or displaced relative to a pillar of the operating table. Such movement can also be detected by means of sensors 98. External impulses, such as jolts against holding arm 1, can also be detected.

FIG. 4 shows a section of holding arm 1, to begin with, but only by way of example. As shown in FIG. 4, two opposite interfaces 500k, 500l, in which two keys 510k, 510l (collectively marked with reference sign 510) are received, are formed in arm segment 20. Keys 510 are each arranged on one operating unit 520k, 520l (collectively marked with reference sign 520). Operating units 520 are used to pick up contact by an operator and to convert such contact into a signal which is converted into a control signal for the brakes by control unit 74. Keys 510k, 510l have an identity which is encoded in the keys. Since the keys are connected to operating units 520k, 520l, keys 510k, 510l also encode the identity of operating units 520k, 520l. Two detecting means 522, 524 for detecting the identity of keys 510k, 510l are also provided in holding arm segment 20. Detecting means 522, 524 are coupled to data bus system 76, as shown in FIG. 3.

According to FIG. 5, operating unit 520 has a substantially flat base member 526, which has an input device 528 with which an operator can enter control signals. Input device 528 is preferably designed as a touch-sensitive surface which is internally connected to a respective microcontroller which can transmit signals via terminal 530 to interface 500, in which a matching second terminal 532 is provided. The detected signals are passed on via terminal 532 from input device 528 to bus system 76 via link 534.

According to this embodiment, key 510 has a substantially cone-shaped body 536, with steps formed on its outside. In addition to terminal 530, key 510 has electrical contacts 538, 539, 540, which can come into contact with matching electrical contacts 542, 543, 544 of interface 500. A chip inside key 510 can be read out via these electrical contacts 538, 539, 540, 542, 543, 544, or detecting means 522 detects the identity of key 510 on the basis of the number and/or kind of closed contacts. Due to the key 510 having the shape of a cone 536, a mechanical coupling is simultaneously provided between operating unit 520 and arm segment 20. In addition to that, a magnetic coupling and/or a detent connection may also be provided.

FIGS. 6 and 7 show further embodiments of the key. Identical and similar elements are marked with the same reference signs, and reference is made in that respect to the entire description of FIGS. 4 and 5 in the foregoing.

FIG. 6 shows arm segment 20, in which interface 500 is formed. A contact is provided on key 510, which in combination with contact 532 allows signal transmission from operating unit 520 to bus system 76. According to this embodiment, key 510 contains visual coding in the form of a QR code 550. The QR code is arranged on an underside 552 of operating unit 520 and can be detected by a respective sensor 554 which is provided in holding arm segment 20. Sensor 554 thus forms detecting means 522. Sensor 554 is coupled by line 556 to bus system 76 in order to transmit the detected identity of key 510 to control unit 74 via bus system 76 (see FIGS. 1 and 2). The control unit then control the releasing device on the basis of the detected barcode 550. It is also possible, using barcode 550, for additional information and data to be captured via sensor 554 or to be transmitted to data bus system 76. Such information may include additional restrictions or degrees of freedom for holding arm 1.

Another preferred variant of key 510 is shown schematically in FIG. 7. Here again, identical and similar elements are marked with the same reference signs, and reference is made in that respect to the entire description of FIGS. 4-6 in the foregoing. Although bus system 76 and contacts 530, 532 are not shown explicitly in FIG. 7, it should be understood that these are nevertheless present in the embodiment shown in FIG. 7.

Key 510 in FIG. 7 has an RFID transponder 558 on operating unit 520, and a matching RFID reader 560 is provided on the arm segment. Reader 560 is coupled to an analyzer 562, which is coupled in turn to bus system 76 (not shown in FIG. 7). The identity of key 510 can be read from RFID transponder 558 when RFID transponder 558 is in the region of reader 560. This is the case when cone 536 is accommodated in its entirety in interface 500, thus producing a mechanical coupling between operating unit 520 and holding arm segment 20. By means of analyzer 562, it is also possible for information and data to be transmitted via reader 560 to operating unit 528. This is particularly advantageous when the latter has a screen or the like, for example, on which data, such as patient data or the like, can be displayed.

In addition, or alternatively, arm segment 20 also has two capacitive sensors 564, as shown in FIG. 8. A gap 570 is provided between projecting sections 566 of operating unit 520 and a surface 558 of arm segment 20. Capacitive sensors 564 are so designed that they detect the slightest changes in distance between a sensor element 572, provided in projecting sections 566, and sensors 564. The cone of key 510 may be slightly elastic, or projecting sections 566 are slightly elastic, so the distance between sensor elements 572 and sensors 564 changes when an operator contacts operating unit 520, as a result of which sensors 564 supply a signal to an analyzer 574, which in turn passes said signal to control unit 74 via data bus system 76. This is an alternative to a touch-sensitive surface 528, as was described with reference to FIG. 5. The advantage of such a configuration is that operating unit 520 is completely sterilisable, in particular autoclavable, because it does not have any sensitive electronic components.

If respective operating units 520 are provided at interfaces 500 (cf. FIG. 1), the operator comes into contact with the two operating units 520 opposite one another by gripping an arm segment 10, 12, 14, 16, 18, 20, 22 (cf. FIG. 1), and the associated joint is released only if there is contact with both the input devices 528, which form the contacting means, of operating unit 520. This means that, when the first arm segment 12 is gripped and the contact is made simultaneously with the two opposite input devices 528, the first joint 13 is released by operating unit 28. In this way, it is possible for the operator to pivot holding arm 1, or arm segments 12-22, about axis A₁. When one or both of the two input devices 528 is let go of, joint 13 is locked again, and pivoting about axis A₁ is no longer possible. If only one of the two input devices 32a, 32b is inadvertently touched, for example by an arm or elbow of the operator, joint 13 is not released and holding arm 1 remains in the locked state and keeps its pose. The same applies accordingly to the other arm segments. For that purpose, operating unit 28 may have a controller or a microprocessor which is adapted to detect contact between input devices 528 and to transmit it in the form of electrical signals.

FIG. 9 shows a holding arm 1 according to a second embodiment of the invention. Holding arm 1 is substantially identical to the one shown in FIGS. 1 and 2 and has seven arm segments 10, 12, 14, 16, 18, 20, 22. Unlike the previous embodiments, holding arm 1 of FIG. 9 has only one interface 500, which is provided in arm segment 20. Contact surfaces may be provided on the other arm segments 10, 12, 14, 16, 18, 22, for example, as disclosed in DE 10 2014 016 824. Alternatively, arm segments 10, 12, 14, 16, 18, 20, 22 can be moved relatively to each other in some other way, for example by active motors in the joints, so that holding arm 1 according to the embodiment shown in FIG. 9 is designed as an active holding arm. Interface 500 is used to receive a key, with a detecting means 528 like the ones disclosed in FIGS. 5-7 being formed at interface 500. Holding arm 1 according to FIG. 9 also includes a control unit and a releasing device, the control unit being designed to control the releasing device according to the identity of the received key.

According to this embodiment, a surgeon can choose an appropriate key 510 and place it in interface 500. Due to the detected identity of key 510, the releasing device is then controlled by means of control unit 76.

1. A holding arm for holding a surgical mechatronic assistance system and/or a surgical instrument, the holding arm comprising:
a proximal end for attaching the holding arm to a base and a distal end for receiving a surgical mechatronic assistance system and/or a surgical instrument;
at least a first and a second arm segment, wherein the first arm segment is connected to a first joint and the second arm segment is connected to a second joint, wherein each joint is configured to be released and locked by a releasing device;
an operating unit for bringing the holding arm into a desired pose;
a control unit coupled to the operating unit and the releasing device, in order to transmit signals from the operating unit to the releasing device;
an interface for receiving a key; and
a detecting means for detecting an identity of the key,
wherein the control unit is configured to control the releasing device according to the identity of the received key.

2. The holding arm of claim 1, wherein the operating unit comprises the interface for receiving the key wherein the key is arranged on an operating unit and the interface is configured to receive the operating unit in a reversibly releasable manner and to receive control signals from the operating unit.

3. The holding arm of claim 1, wherein the key is a data key or a software key.

4. The holding arm of claim 1, wherein the control unit is configured to control the releasing device such that when a first identity of a first key is detected, a first plurality of restrictions for the holding arm is provided, and when a second identity of a second key is detected, a second plurality of restrictions for the holding arm is provided.

5. The holding arm of claim 1, wherein the detecting means has an optical detecting means for optically detecting the identity of the key.

6. The holding arm of claim 5, wherein the optical detecting means is configured to detect colour coding and/or structural coding of the identity of the key.

7. The holding arm of claim 1, wherein the detecting means comprises a receiver for receiving electromagnetic waves, wherein the electromagnetic waves represent the identity of the key.

8. The holding arm of claim 1, wherein the detecting means comprises an electronic interface for receiving an identification signal from the key.

9. The holding arm of claim 1, wherein the detecting means comprises an electromagnetic lock for receiving the key.

10. The holding arm of claim 1, further comprising a sterile bag surrounding the holding arm, wherein the sterile bag has a recess and/or can be penetrated in the region of the interface for receiving an operating unit in a reversibly releasable manner.

11. The holding arm of claim 1, wherein a surgical mechatronic assistance system is received at the distal end and an operating unit and/or the interface is arranged on the assistance system.

12. An operating unit for the holding arm of claim 1, the operating unit comprising a key to be received by the interface.

13. The operating unit of claim 12, further comprising,
a base member;
an input device with which an operator can enter a control signal; and
an interface for transmitting the control signal to the holding arm,
wherein the key specifies the identity of the operating unit.

14. The operating unit of claim 12, wherein the key comprises visual coding.

15. The operating unit of claim 14, wherein the visual coding comprises colour coding or structural coding.

16. The operating unit of claim 12, wherein the key comprises a transmitter for transmitting electromagnetic waves.

17. The operating unit of claim 12, wherein the key comprises an electronic interface for transmitting an identification signal.

18. The operating unit of claim 12, wherein the key is a mechanically insertable key.

19. The operating unit of claim 12, further comprising an interface for an optical reflector for surgical navigation.

20. The operating unit of claim 12, further comprising display devices, for displaying a status of the operating unit, of the holding arm, of a mechatronic assistance system, and/or of a surgical instrument attached to the holding arm.

21. The operating unit having of claim 12, further comprising a screen for displaying data and/or a model of a patient.

22. The operating unit of claim 12, further comprising a vibration module configured to vibrate the operating unit.

23. The operating unit of claim 13, wherein the input device has a touch-sensitive area for controlling the holding arm.

24. The operating unit of claim 12, further comprising an electronic coil for surgical navigation.

25. The operating unit of claim 13, wherein the input device comprises a capacitive sensor configured to detect a change in the distance of the operating unit from the holding arm and to convert said change into an electrical signal.
